(19) United States
(12) Patent Application Publication
(10) Pub. No.: US 2022/0160776 A1
(43) Pub. Date: May 26, 2022

(54) METHOD FOR TREATING OR PREVENTING GASTROINTESTINAL BLEEDING

(71) Applicant: MESOBLAST INTERNATIONAL SÁRL, Meyrin (CH)

(72) Inventors: Silviu ITESCU, Melbourne (AU); Kenneth M. BOROW, New York, NY (US)

(21) Appl. No.: 17/425,893

(22) PCT Filed: Jan. 28, 2020
(86) PCT No.: PCT/IB2020/050662

§ 371 Date: Jul. 26, 2021

Related U.S. Application Data
(60) Provisional application No. 62/797,655, filed on Jan. 28, 2019.

(51) Int. Cl. A61K 35/28; A61P 1/00

(52) U.S. Cl. CPC A61K 35/28; A61P 1/00

(57) ABSTRACT

The present disclosure relates to method of treating or preventing gastrointestinal (GI) bleeding in a subject in need thereof, the method comprising administering to the subject a composition comprising mesenchymal lineage precursor or stem cells (MLPSCs).

### FIELD OF THE DISCLOSURE

The present disclosure relates to methods for treating or preventing gastrointestinal bleeding in a subject in need thereof.

### BACKGROUND

In the United States, there are approximately 250,000-300,000 patients annually who suffer from advanced systolic heart failure (NYHA Class IIIb-IV) who despite optimal medical therapy (excluding mechanical assist devices) have a one-year mortality >25% and exceeding 50% in class IV patients. The only options to increase survival in these patients are the use of cardiac assist devices or heart transplants. Due to the decline in organ donations and limited availability of healthy donor hearts, the treatment of CHF with mechanical circulatory support devices such as LVADs is gaining momentum, with 4,500-5,500 assist devices implanted annually in the United States. However, rehospitalization is frequent in patients with an LVAD ranging from 2.1-2.7 times per year. The majority of patients rehospitalized for non-device related causes are as a result of gastrointestinal bleeding (34%-44%) and infections (36%-44%) (see, for example, Chatterjee A, Feldmann C, Hanke J S (2018) The momentum of HeartMate 3: a novel active magnetically levitated centrifugal left ventricular assist device (LVAD). J Thorac Dis 10 (Suppl 15): S1790-S1793; and Mehra, M R Salerno C, Cleveland J C (2018) Health care resources use and cost implications in the MOMENTUM 3 long-term outcome study: a randomized controlled trial of a magnetically levitated cardiac pump in advanced heart failure).

Accordingly, methods for treating or preventing gastrointestinal bleeding are required.

### SUMMARY OF THE DISCLOSURE

Preclinical cardiac studies performed with mesenchymal lineage precursor cells have demonstrated unexpectedly that administration of the cells to patients with left ventricular assisted device complications leads to a reduction in gastrointestinal (GI) bleeding and related hospitalization.

The present disclosure therefore provides a method of treating or preventing gastrointestinal (GI) bleeding in a subject in need thereof, the method comprising administering to the subject a composition comprising mesenchymal lineage precursor or stem cells (MLPSCs).

In one example the GI bleeding is caused by complications arising from LVAD implantation in the subject.

In one example the GI bleeding is associated with inflammation arising from LVAD implantation in the subject.

In one example the LVAD implantation is a bridge to transplant (BTT) or destination therapy (DT).

In one example the subject is undergoing treatment or has been treated for heart failure. The heart failure may be due, for example, to hypertension, cardiomyopathy (ischemic or non-ischemic), myocarditis, obesity, or diabetes.

The method according to any preceding claim, wherein the subject has an inflamed myocardium.

In another example, the GI bleeding is associated with aortic stenosis.

In another example, the GI bleeding is associated with von Willebrand's disease.

In another example, the GI bleeding is associated with epistaxis.

In another disorder, the GI bleeding is associated with hemorrhoids, peptic ulcers, tears or inflammation in the esophagus, diverticulosis, diverticulitis, ulcerative colitis, Crohn's disease, colonic polyps, or cancer in the colon, stomach or esophagus.

In another example, the method comprises the steps of:

i) selecting a subject showing signs of GI bleeding or at risk of developing GI bleeding, and

ii) administering to the subject a population of mesenchymal lineage stem or precursor cells and/or progeny thereof and/or soluble factors derived therefrom.

In one example the subject is at risk of developing GI due to an implanted LVAD.

In one example, the cells, progeny thereof or soluble factors are administered to the subject by a catheter-based system. In a further example, the cells, and/or progeny thereof and/or soluble factors are administered to the subject's myocardium at or near the site of tissue damage using a catheter inserted into the subject's venous system. In another example, the cells, and/or progeny thereof and/or soluble factors may be administered systemically. The delivery of cells, and/or progeny thereof and/or soluble factors may be performed after identifying a region of the myocardium in need of treatment.

In a further example, the cells, and/or progeny thereof and/or soluble factors are administered to the subject by transendocardial injection, intracoronary infusion, transepicardial injection or via a trans-atrial septal or coronary sinus approach.

In another example, the mesenchymal lineage stem or precursor cells and/or progeny thereof and/or soluble factors derived therefrom are administered to the subject following acute myocardial infarction. In a further example, the mesenchymal lineage stem or precursor cells and/or progeny thereof and/or soluble factors derived therefrom are administered to the subject between about 1 and 7 days following diagnosis of heart failure. In a further example, the mesenchymal lineage stem or precursor cells and/or progeny thereof and/or soluble factors derived therefrom are administered to the subject between about 1 and 7 days following myocardial infarction. In a further example, the mesenchymal lineage stem or precursor cells and/or progeny thereof and/or soluble factors derived therefrom are administered to the subject between about 3 and 5 days following diagnosis or heart failure, or following myocardial infarction.

In some embodiments, the methods of the present disclosure also comprise administering to the heart or to the subject, a population of mesenchymal lineage stem or precursor cells enriched for STRO-1⁺ cells and/or progeny thereof and/or soluble factors derived therefrom. In another example, the methods of the present disclosure comprise administering to the heart or to the subject, a population of mesenchymal lineage stem or precursor cells enriched for STRO-^{bright} cells and/or progeny thereof and/or soluble factors derived therefrom.

In another example, the population of mesenchymal lineage stem or precursor cells express tissue non-specific alkaline phosphatase (TNAP), and/or the progeny cells and/or soluble factors are derived from mesenchymal lineage cells that express TNAP. In another example, the population of mesenchymal lineage stem or precursor cells express angiopoietin-1 (Ang1) in an amount of at least 0.1 μg/10⁶ cells and/or the progeny cells and/or soluble factors are derived from mesenchymal lineage precursor cells that express Ang1 in an amount of at least 0.1 μg/10⁶ cells. In another example, the population of mesenchymal lineage stem or precursor cells express Ang1 in an amount of at least 0.5 μg/10⁶ cells. In another example, the population of mesenchymal lineage stem or precursor cells express Ang1 in an amount of at least 1 μg/10⁶ cells. In another example, the population of mesenchymal lineage stem or precursor cells express Vascular Endothelial Growth Factor (VEGF) in an amount less than about 0.05 μg/10⁶ cells and/or the progeny cells and/or soluble factors are derived from mesenchymal lineage precursor cells that express VEGF in an amount less than about 0.05 μg/10⁶ cells and/or the progeny cells. In another example, the population of mesenchymal lineage stem or precursor cells express VEGF in an amount less than about 0.03 μg/10⁶ cells. In another example, the population of mesenchymal lineage stem or precursor cells express Ang1:VEGF at a ratio of at least about 2:1 and/or the progeny cells and/or soluble factors are derived from mesenchymal lineage stem or precursor cells that express Ang1:VEGF at a ratio of at least about 2:1. In another example, the population of mesenchymal lineage stem or precursor cells express Ang1:VEGF at a ratio of at least about 10:1. In another example, the population of mesenchymal lineage stem or precursor cells express Ang1:VEGF at a ratio of at least about 20:1. In another example, the population of mesenchymal lineage stem or precursor cells express Ang1:VEGF at a ratio of at least about 30:1.

In one example, the population of mesenchymal lineage stem or precursor cells and/or progeny thereof are administered to the heart or to the subject in a therapeutically effective amount.

In one example, the population of mesenchymal lineage stem or precursor cells and/or progeny thereof are administered to the heart or to the subject over multiple doses. In a further example, the methods of the present disclosure comprise administering from 1×10⁶ to 8×10⁸ cells. In another example, the methods of the present disclosure comprise administering about 1.5×10⁸ cells.

In one example, the mesenchymal lineage stem or precursor cells and/or progeny cells thereof and/or soluble factors derived therefrom are administered in the form of a composition comprising the mesenchymal lineage stem or precursor cells and/or progeny cells thereof and/or soluble factors derived therefrom together with a pharmaceutically acceptable carrier and/or excipient. In a further example, the population of mesenchymal lineage stem or precursor cells and/or progeny thereof have been expanded in culture prior to administration and/or prior to obtaining the soluble factors.

In a further example according to any method or use described herein, the population of mesenchymal lineage precursor cells and/or progeny thereof are isolated or purified.

In a further example, the population of mesenchymal lineage stem or precursor cells and/or progeny thereof are derived from a donor subject. The donor subject may be the same subject into which the cells, and/or progeny thereof and/or soluble factors derived therefrom are administered in which case the cells are autologous. In another example, the donor subject is a different subject into which the cells, and/or progeny thereof and/or soluble factors derived therefrom are administered in which case the cells are allogeneic.

The present disclosure also provides a kit comprising a population of mesenchymal lineage stem or precursor cells and/or progeny thereof and/or soluble factors derived therefrom or a composition described herein and a delivery device for administration of the cells, and/or progeny thereof and/or soluble factors. In one example, the delivery device is a catheter.

In another example, the subject according to the present disclosure is a mammal.

In a further example, the subject is a human, including an adolescent human or pediatric human. In a particular example the subject is greater than or equal to 18 years of age.

In a further example, the subject has had a heart failure event in the twelve months preceding administration of the population of mesenchymal lineage stem or precursor cells and/or progeny thereof and/or soluble factors derived therefrom.

### BRIEF DESCRIPTION OF THE DRAWINGS

FIG. 1: Reduced Cumulative Incidence and Delay of Onset of Non-surgical Major GI Bleeding in the 30 Patient Pilot Trial

FIG. 2: Trial Design for the 159 Patient Confirmatory Trial

FIG. 3: Reduced Cumulative Incidence and Delay of Onset of Non-surgical Major GI Bleeding at 6 Months in the 159 Patient Confirmatory Trial

FIG. 4: Comparison of Decreased Rate of Non-surgical Major GI Bleeding Events at 6 Months per 100 Patient-Months of Follow-up for the 30 Patient Pilot Trial and the 159 Patient Confirmatory Trial

FIG. 5: Hospitalization Rate for Non-surgical Major GI Bleeding Events at 6 Months per 100 Patient-Months of Follow-up for the 159 Patient Confirmatory Trial

### DETAILED DESCRIPTION

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

Unless specifically defined otherwise, all technical and scientific terms used herein shall be taken to have the same meaning as commonly understood by one of ordinary skill in the art (e.g., in cell culture, molecular genetics, stem cell differentiation, immunology, immunohistochemistry, protein chemistry, and biochemistry).

Unless otherwise indicated, the surgical techniques utilized in the present disclosure are standard procedures, well known to those skilled in the art.

Methods of obtaining and enriching a population of mesenchymal lineage stem or precursor cells are known in the art. For example, enriched populations of mesenchymal lineage stem or precursor cells can be obtained by the use of flow cytometry and cell sorting procedures based on the use of cell surface markers that are expressed on mesenchymal lineage stem or precursor cells.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### Selected Definitions

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the singular form “a”, “an” and “the” include singular and plural references unless the context indicates otherwise.

As used herein, the term “heart failure” may be used interchangeably with the term “congestive heart failure (CHF)” and refers to a condition in which the heart cannot pump enough blood to the body's other organs due to, for example, heart muscle malfunction, weakening of the heart muscle, referred to as “cardiomyopathy” and other heart muscle related reasons. Congestive heart failure is characterised, among other effects, by left ventricle (LV) chamber dilation, decrease LV contractility and elevated levels of circulating catecholamines. In another example, heart failure occurs due to ischemic and other reperfusion, and other non-ischemic factors. Heart failure includes, but is not limited to the following symptoms or signs individually or collectively: cardiac reperfusion injury, compensated hypertrophy, human end stage heart failure, hypertensive cardiomyopathy, left ventricular hypertension, left or right ventricular dilation, left or right ventricular failure, maladaptive hypertrophy, myocardial structural disarrangement (apoptosis and loss of cardiomyocytes) and myocardial dysfunction (loss in contraction and/or relaxation) and pressure overloaded heart.

By “isolated” or “purified” it is meant a cell which has been separated from at least some components of its natural environment. This term includes gross physical separation of the cells from its natural environment (e.g. removal from a donor). The term “isolated” includes alteration of the cell's relationship with the neighbouring cells with which it is in direct by, for example, dissociation. The term “isolated” does not refer to a cell which is in a tissue section. When used to refer to the population of cells, the term “isolated” includes populations of cells which result from proliferation of the isolated cells of the disclosure.

As used herein, the term “left ventricular hypertension (LVH)” is a condition wherein the cardiac muscle responds to increased resistance in the circulation by becoming enlarged. However, with time, the fibers of the hypertrophied heart muscle become thickened and shortened and consequently less able to relax. Hypertension makes the myocardium work harder. The resulting hypertrophy is the product of the thickening or shortening of the muscle fibers of the heart. Under these conditions, it becomes more difficult for the heart to relax and go through the normal cycle of contraction and relaxation. Changes in the myocardium appear in the collagen resulting in increased stiffness. The outcome of this process is a heart that is less able to meet the output demands of normal circulation.

As used herein, the term “left ventricular dilation” refers to a left ventricular enlargement, which is a compensatory process to help maintain an adequate amount of blood that is ejected from the ventricle, temporarily improving cardiac output. However, with time, this increase in size of the ventricle cavity however also results in a reduction of the percentage of left ventricular volume of blood that is ejected (called ejection fraction) and has significant physiological implications. Left ventricular dilation is a well-recognised precursor and sign of ventricular dysfunction and congestive heart failure after myocardial infarction. Similarly, right ventricular dilation refers to a right ventricular enlargement and associated signs or disorder.

As used herein, the term “left ventricular failure” refers to a disorder where the left side of the heart fails to pump blood effectively. This results in a back-flow, pressure and/or congestion of blood into the lungs. Signs indicating left ventricular failure include a laterally displaced apex beat. A gallop rhythm may be heard as a marker of increased blood flow or increased intra-cardiac pressure.

As used herein, the term “cardiomyopathy” refers to a condition in which the heart muscle (the myocardium) becomes inflamed and enlarged. Several different types of cardiomyopathy are known in the art, including dilated cardiomyopathy in which the heart muscle is stretched and becomes thinner, hypertrophic cardiomyopathy in which the heart muscle cells enlarge and cause the walls of the heart to thicken, and restrictive cardiomyopathy in which the heart becomes stiff and rigid because of abnormal tissue e.g. scar tissue.

As used herein, the term “myocardial infarction” is also understood as referring to a heart attack. Heart attack occurs when blood stops flowing properly to a part of the heart and the heart muscle is injured because it is not receiving enough oxygen. This can occur when one of the coronary arteries that supplies blood to the heart develops a blockage.

As used herein, the terms “treating”, “treat” or “treatment” include administering a population of mesenchymal lineage stem or precursor cells and/or progeny thereof and/or soluble factors derived therefrom to thereby reduce or eliminate at least one symptom of heart failure. In one particular example, the treatment reduces the LVESV value by at least 5%, at least 10%, at least 15%, at least 20%, at least 25%, at least 30%, at least 35%, at least 40%, at least 45%, or at least 50% compared to the baseline value (i.e. before administration of mesenchymal lineage stem or precursor cells and/or progeny thereof and/or soluble factors derived therefrom).

The term “prevent” or “preventing” as used herein include administering a population of mesenchymal lineage stem or precursor cells and/or progeny thereof and/or soluble factors derived therefrom to thereby stop or hinder the development of at least one symptom of heart failure.

The term “subject” as used herein refers to a mammal, including but not limited to murines, rats, simians, humans, domestic and farm animals.

As used herein, the term “genetically unmodified” refers to cells that have not been modified by transfection with a nucleic acid. For the avoidance of doubt, in the context of the present disclosure a mesenchymal lineage precursor or stem cell transfected with a nucleic acid encoding Angl would be considered genetically modified.

Mesenchymal Lineage Precursor Cells

As used herein, the term “mesenchymal lineage precursor or stem cell” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp.

In one example the mesenchymal lineage precursor cells are STRO-1+ mesenchymal precursor cells (MPCs). As used herein, the phrase “STRO-1+ multipotential cells” shall be taken to mean STRO-1+ and/or TNAP+ progenitor cells capable of forming multipotential cell colonies.

STRO-1+ multipotential cells are cells found in bone marrow, blood, dental pulp cells, adipose tissue, skin, spleen, pancreas, brain, kidney, liver, heart, retina, brain, hair follicles, intestine, lung, lymph node, thymus, bone, ligament, tendon, skeletal muscle, dermis, and periosteum; and are capable of differentiating into germ lines such as mesoderm and/or endoderm and/or ectoderm. Thus, STRO-1+ multipotential cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

Mesenchymal lineage precursor or stem cells can be isolated from host tissues and enriched for by selection of STRO-1+ cells. For example, a bone marrow aspirate from a subject may be further treated with an antibody to STRO-1 or TNAP to enable selection of mesenchymal lineage precursor or stem cells. In one example, the mesenchymal lineage precursor or stem cells can be enriched for by using the STRO-1 antibody described in (Simmons & Torok-Storb, 1991).

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for STRO-1+ cells comprises at least about 0.1% or 0.5% or 1% or 2% or 5% or 10% or 15% or 20% or 25% or 30% or 50% or 75% STRO-1+ cells. In this regard, the term “population of cells enriched for STRO-1+ cells” will be taken to provide explicit support for the term “population of cells comprising X% STRO-1+ cells”, wherein X% is a percentage as recited herein. The STRO-1+ cells can, in some examples, form clonogenic colonies, e.g. CFU-F (fibroblasts) or a subset thereof (e.g., 50% or 60% or 70% or 70% or 90% or 95%) can have this activity.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+).

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+ (HSP-90β), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

In one example, the STRO-1+ cells are STRO-1^{bright} (syn. STRO-1^{bright}). In another example, the STRO-1^{bright} cells are preferentially enriched relative to STRO-1^{dim} or STRO-1^{intermediate} cells.

In another example, the STRO-1^{bri} cells are additionally one or more of TNAP+, VCAM-1+, THY-1+, STRO-2+, STRO-4+ (HSP-90β) and/or CD146+. For example, the cells are selected for one or more of the foregoing markers and/or shown to express one or more of the foregoing markers. In this regard, a cell shown to express a marker need not be specifically tested, rather previously enriched or isolated cells can be tested and subsequently used, isolated or enriched cells can be reasonably assumed to also express the same marker.

In one example, the mesenchymal precursor cells (MPCs) are perivascular mesenchymal precursor cells as defined in WO 2004/85630 (which is incorporated herein by reference in its entirety) characterised by the presence of the perivascular marker 3G5. For example, the MPCs express a marker of a perivascular cell, e.g., the cells are STRO-1+ or STRO-1^{bri} and/or 3G5+. In one example, the cells are or were previously or are progeny of cells that were isolated from vascularized tissue or organs or parts thereof.

A cell that is referred to as being “positive” for a given marker may express either a low (lo or dim) or a high (bright, bri) level of that marker depending on the degree to which the marker is present on the cell surface, where the terms relate to intensity of fluorescence or other marker used in the sorting process of the cells. The distinction of lo (or dim or dull) and bri will be understood in the context of the marker used on a particular cell population being sorted. A cell that is referred to as being “negative” for a given marker is not necessarily completely absent from that cell. This term means that the marker is expressed at a relatively very low level by that cell, and that it generates a very low signal when detectably labelled or is undetectable above background levels, e.g., levels detected using an isotype control antibody.

The term “bright” or “bri” as used herein, refers to a marker on a cell surface that generates a relatively high signal when detectably labelled. Whilst not wishing to be limited by theory, it is proposed that “bright” cells express more of the target marker protein (for example the antigen recognized by STRO-1) than other cells in the sample. For instance, STRO-1^{bri} cells produce a greater fluorescent signal, when labelled with a FITC-conjugated STRO-1 antibody as determined by fluorescence activated cell sorting (FACS) analysis, than non-bright cells (STRO-1^{dull/dim}). In one example, “bright” cells constitute at least about 0.1% of the most brightly labeled bone marrow mononuclear cells contained in the starting sample. In other examples, “bright” cells constitute at least about 0.5%, at least about 1%, at least about 1.5%, or at least about 2%, of the most brightly labelled bone marrow mononuclear cells contained in the starting sample. In an example, STRO-1bright cells have 2 log magnitude higher expression of STRO-1 surface expression relative to “background”, namely cells that are STRO-1−. By comparison, STRO-1dim and/or STRO-1intermediate cells have less than 2 log magnitude higher expression of STRO-1 surface expression, typically about 1 log or less than “background”.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium.

In another example, the mesenchymal lineage precursor or stem cells are CD29+, CD54+, CD73+, CD90+, CD102+, CD105+, CD106+, CD166+, MHC1+ MSCs (e.g. remestemcel-L).

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

Mesenchymal lineage precursor or stem cells may also be cryopreserved prior to administration to a subject.

### Modification of the Cells

In one embodiment, the mesenchymal lineage precursor or stem cells are genetically unmodified. In one example, mesenchymal lineage precursor or stem cells of the present disclosure may be genetically modified or genetically unmodified and express Ang1 in an amount of at least 0.1 μg/10⁶ cells. For example, the mesenchymal lineage stem or precursor cells can express Ang1 in an amount of at least 0.1 μg/10⁶ cells, 0.2 μg/10⁶ cells, 0.3 μg/10⁶ cells, 0.4 μg/10⁶ cells, 0.5 μg/10⁶ cells, 0.6 μg/10⁶ cells, 0.7 μg/10⁶ cells, 0.8 μg/10⁶ cells, 0.9 μg/10⁶ cells, 1 μg/10⁶ cells, 1.1 μg/10⁶ cells, 1.2 μg/10⁶ cells, 1.3 μg/10⁶ cells, 1.4 μg/10⁶ cells, 1.5 μg/10⁶ cells.

In an example, the mesenchymal lineage precursor or stem cells of the present disclosure are genetically unmodified and express Ang1 in an amount of at least 0.1 μg/10⁶ cells. In other examples, the cells may express Ang1 in an amount of at least 0.2 μg/10⁶ cells, 0.3 μg/10⁶ cells, 0.4 μg/10⁶ cells, 0.5 μg/10⁶ cells, 0.6 μg/10⁶ cells, 0.7 μg/10⁶ cells, 0.8 μg/10⁶ cells, 0.9 μg/10⁶ cells, 1 μg/10⁶ cells, 1.1 μg/10⁶ cells, 1.2 μg/10⁶ cells, 1.3 μg/10⁶ cells, 1.4 μg/10⁶ cells, 1.5 μg/10⁶ cells.

In another aspect, the mesenchymal lineage precursor or stem cells of the present disclosure express VEGF in an amount less than about 0.05 μg/10⁶ cells. However, in various embodiments it is envisaged that the mesenchymal lineage stem or precursor cells of the present disclosure may express VEGF in an amount less than about 0.05 μg/10⁶ cells, 0.04 μg/10⁶ cells, 0.03 μg/10⁶ cells, 0.02 μg/10⁶ cells, 0.01 μg/10⁶ cells, 0.009 μg/10⁶ cells, 0.008 μg/10⁶ cells, 0.007 μg/10⁶ cells, 0.006 μg/10⁶ cells, 0.005 μg/10⁶ cells, 0.004 μg/10⁶ cells, 0.003 μg/10⁶ cells, 0.002 μg/10⁶ cells, 0.001 μg/10⁶ cells.

In an example, the mesenchymal lineage precursor or stem cells of the present disclosure are genetically unmodified and express VEGF in an amount less than about 0.05 μg/10⁶ cells. However, in various embodiments of this example, it is envisaged that the mesenchymal lineage precursor or stem cells of the present disclosure may express VEGF in an amount less than about 0.05 μg/10⁶ cells, 0.04 μg/10⁶ cells, 0.03 μg/10⁶ cells, 0.02 μg/10⁶ cells, 0.01 μg/10⁶ cells, 0.009 μg/10⁶ cells, 0.008 μg/10⁶ cells, 0.007 μg/10⁶ cells, 0.006 μg/10⁶ cells, 0.005 μg/10⁶ cells, 0.004 μg/10⁶ cells, 0.003 μg/10⁶ cells, 0.002 μg/10⁶ cells, 0.001 μg/10⁶ cells.

The amount of cellular Ang1 and/or VEGF that is expressed in a composition or culture of mesenchymal lineage precursor or stem cells may be determined by methods known to those skilled in the art. Such methods include, but are not limited to, quantitative assays such as quantitative ELISA assays, for example or fluorescence-linked immunosorbent assay (FLISA), Western blot, competition assay, radioimmunoassay, lateral flow immunoassay, flow-through immunoassay, electrochemiluminescent assay, nephelometric-based assays, turbidometric-based assay, fluorescence activated cell sorting (FACS)-based assays for detection of Ang-1 or VEGF in culture medium used to culture mesenchymal lineage precursor cells or stem cells, and surface plasmon resonance (SPR or Biacore).

It is to be understood, however, that the scope of the present disclosure is not to be limited to any particular method for determining the amount or level of Ang1 or VEGF expressed in the mesenchymal lineage precursor or stem cells of the present disclosure.

In one example the level of Ang1 or VEGF expressed by a composition or culture of mesenchymal lineage precursor or stem cells is determined by an ELISA assay. In such an assay, a cell lysate from a culture of mesenchymal lineage precursor or stem cells is added to a well of an ELISA plate. The well may be coated with a primary antibody, either a monoclonal or a polyclonal antibody(ies), against the Ang1 or VEGF. The well then is washed, and then contacted with a secondary antibody, either a monoclonal or a polyclonal antibody(ies) , against the primary antibody. The secondary antibody is conjugated to an appropriate enzyme, such as horseradish peroxidase, for example. The well then may be incubated, and then is washed after the incubation period. The wells then are contacted with an appropriate substrate for the enzyme conjugated to the secondary antibody, such as one or more chromogens. Chromogens which may be employed include, but are not limited to, hydrogen peroxide and tetramethylbenzidine. After the substrate(s) is (are) added, the well is incubated for an appropriate period of time. Upon completion of the incubation, a “stop” solution is added to the well in order to stop the reaction of the enzyme with the substrate(s). The optical density (OD) of the sample then is measured. The optical density of the sample is correlated to the optical densities of samples containing known amounts of Ang1 or VEGF in order to determine the amount of Ang1 or VEGF expressed by the culture of mesenchymal lineage precursor or stem cells being tested.

In another aspect, the mesenchymal lineage precursor or stem cells of the present disclosure express Ang1:VEGF at a ratio of at least about 2:1. However, in various embodiments it is envisaged that the mesenchymal lineage precursor or stem cells of the present disclosure may express Ang1:VEGF at a ratio of at least about 10:1, 15:1, 20:1, 21:1, 22:1, 23:1, 24:1, 25:1, 26:1, 27:1, 28:1, 29:1, 30:1, 31:1, 32:1.

In one example, the mesenchymal lineage precursor or stem cells of the present disclosure are genetically unmodified and express Ang1:VEGF at a ratio of at least about 2:1. However, in various embodiments it is envisaged that the mesenchymal lineage precursor or stem cells of the present disclosure may express Ang1:VEGF at a ratio of at least about 10:1, 15:1, 20:1, 21:1, 22:1, 23:1, 24:1, 25:1, 26:1, 27:1, 28:1, 29:1, 30:1, 31:1, 32:1.

Methods for determining the Ang1:VEGF expression ratio will be apparent to one of skill in the art. In an example of a method of determining a ratio of Ang 1 and VEGF expression, Ang1 and VEGF expression levels are quantitated via quantitative ELISA as discussed above. In such an example, after quantifying the levels of Ang1 and VEGF, a ratio based on the quantitated levels of Ang1 and VEGF could be represented as: (level of Ang1/level of VEGF)=Ang1:VEGF ratio.

The mesenchymal lineage precursor or stem cells of the present disclosure may be altered in such a way that upon administration, lysis of the cell is inhibited. Alteration of an antigen can induce immunological non-responsiveness or tolerance, thereby preventing the induction of the effector phases of an immune response (e.g., cytotoxic T cell generation, antibody production etc.) which are ultimately responsible for rejection of foreign cells in a normal immune response. Antigens that can be altered to achieve this goal include, for example, MHC class I antigens, MHC class II antigens, LFA-3 and ICAM-1.

In another example, the mesenchymal lineage precursor or stem cells may be genetically modified to express an gene product to be supplied to the subject receiving the transplantation. Examples of gene products that can be delivered to a subject via genetically modified mesenchymal lineage precursor cells include gene products that can prevent future cardiac disorders, such as growth factors which encourage blood vessels to invade the heart muscle (e.g. vascular endothelial growth factor (VEGF), fibroblast growth factor (FGF), transforming growth factor beta (TGF-β) and angiotensin).

The mesenchymal lineage precursor or stem cells may also be genetically modified to express proteins of importance for the differentiation and/or maintenance of striated skeletal muscle cells. Exemplary proteins include growth factors (TGF-β, insulin-like growth factor 1 (IGF-1), FGF), myogenic factors (e.g. myoD, myogenin, myogenic factor 5 (Myf5), myogenic regulatory factor (MRF)), transcription factors (e.g. GATA-4), cytokines (e.g. cardiotropin-1), members of the neuregulin family (e.g. neuregulin 1, 2 and 3) and homeobox genes (e.g. Csx, tinman and NKx family).

### Heart Failure

Heart failure occurs when the heart is unable to pump sufficiently to maintain blood flow to meet the needs of the body. One cause of heart failure is myocardial infarction (MI). A MI occurs when blood stops flowing properly to a part of the heart. The lack of blood supply results in a localized area of myocardial necrosis referred to as an infarct or infarction. The infarcted heart is unable to pump sufficiently to maintain blood flow to meet the needs of the body leading to heart failure. Post-MI, a series of compensatory mechanisms are initiated, serving to buffer the fall in cardiac output and assisting to maintain sufficient blood pressure to perfuse the vital organs. As a result, patients with heart failure may not progress for extended periods of time. However, the compensatory mechanisms eventually fail to compensate for the damaged heart, resulting in a progressive decline in cardiac output, termed “progressive heart failure”.

A diagnosis of MI is created by integrating the history of the presenting illness and physical examination with electrocardiogram finding and cardiac markers. A coronary angiogram can be performed which allows visualisation of narrowings or obstructions on the heart vessels. According to WHO criteria as revised in 2000 (Alpert J S, Thygesen K, Antman E, Bassand J P. (2000). “Myocardial infarction redefined—a consensus document of The Joint European Society of Cardiology/American College of Cardiology Committee for the redefinition of myocardial infarction”. J Am Coll Cardiol 36 (3): 959-69), a cardiac troponin rise accompanied by either typical symptoms, pathological Q waves, ST elevation or depression or coronary intervention are diagnostic of MI.

For more than 70 years, the 12-lead electrocardiogram (ECG) has remained the standard for determining the presence and location of MIs. It is universally available, noninvasive, inexpensive and easily repeatable. The quantitative Selvester QRS scoring system (Selvester R H et al. (1985) Arch Intern Med 145(10):1877-1881) which was designed from computer simulations, utilises the information on the ECG to estimate MI size. The Selvester scoring system is a 50 criteria 31 point QRS scoring system based on observations of Q- and R-wave durations and R/Q and R/S amplitude ratios in the standard 12-lead ECG. Methods for determining infarct size, including, but not limited to, QRS scoring are familiar to persons skilled in the art.

Cardiac markers can also be measured to determine incidence of MI. Such markers include troponins T and I, creatinine kinase, myoglobin levels, natriuretic peptides (e.g. B-type natriuretic peptide), C-reactive protein (CRP), erythrocyte sedimentation rate (ESR), heart type fatty acid binding protein and copeptin, mid-regional pro-atrial natriuretic peptide, ST2, C-terminal pro-endothelin 1, and mid-regional pro-adrenomedullin.

Troponin is a protein released from myocytes when irreversible myocardial damage occurs. It is highly specific to cardiac tissue and accurately diagnoses myocardial infarction with a history of ischaemic pain or ECG changes reflecting ischaemia. Cardiac troponin level is dependent on infarct size, thus providing an indicator for the prognosis following an infarction.

### Catheter-Based Delivery Systems

Any catheter-based delivery system that allows for the injection of mesenchymal lineage precursor or stem cells and/or progeny thereof and/or soluble factor derived therefrom, or compositions comprising same into a subject's myocardium or at a site near the area of cardiac tissue damage can be used in the practice of the methods of the present disclosure. In certain examples, the catheter is introduced percutaneously (e.g., into the femoral artery or another blood vessel) and routed through the vascular system to the subject's myocardium where it is used to deliver the mesenchymal lineage precursor or stem cells and/or progeny thereof and/or soluble factor derived therefrom, or compositions comprising same via a needle that is extruded from the end of the catheter. In other examples, the catheter reaches the heart through minimal surgical incision (e.g., limited thoracotomy, which involves an incision between the ribs).

Several catheters have been designed in order to precisely deliver agents to a damaged region within the heart, for example, an infarct region (see, for example, U.S. Pat. Nos. 6,102,926; 6,120,520; 6,251,104; 6,309,370; 6,432,119, and 6,485,481, each of which is incorporated herein by reference in its entirety). The catheter may be guided to the indicated location by being passed down a steerable or guidable catheter having an accommodating lumen (see, for example, U.S. Pat. No. 5,030,204, which is incorporated herein by reference in its entirety) or by means of a fixed configuration guide catheter (see, for example, U.S. Pat. No. 5,104,393, which is incorporated herein by reference in its entirety) Alternatively, the catheter may be advanced to the desired location within the heart by means of a deflectable stylet (see, for example WO 93/04724, which is incorporated herein by reference in its entirety), or a deflectable guide wire (see, for example, U.S. Pat. No. 5,060,660, which is incorporated herein by reference in its entirety).

The catheter may be coupled to a cardiac mapping system, which allows determination of the location and extent of the damaged/defective zone(s) (as described above). Once an area in need of treatment is identified, the steering guide may be pulled out leaving the needle at the site of injection. Part or all of the mesenchymal lineage precursor or stem cells and/or progeny thereof and/or soluble factor derived therefrom is sent down the lumen of the catheter and injected into the myocardium. The catheter is retracted from the subject when all the injections have been performed.

The needle element may be ordinarily retracted within a sheath at the time of guiding the catheter into the subject's heart to avoid damage to the venous system and/or the myocardium. At the time of injection, the needle is extruded from the tip of the catheter. During injection, the needle protrudes less than 10 mm, less than 7.5 mm or less than 5 mm into an adult heart muscle wall. Depending on the site of injection, the maximum length may be altered. For infants and children, the protrusion depth is correspondingly less, as determined by the actual or estimated wall thickness. The needle gauge used in transplantation of the cells can be, for example, 25 to 30.

In one example, the catheter used to deliver the mesenchymal lineage precursor or stem cells and/or progeny thereof and/or soluble factor derived therefrom to the myocardium is configured to include a feedback sensor for mapping the penetration depth and location of the needle insertion. The use of a feedback sensor provides the advantage of accurately targeting the injection location. The target location for delivering the cell composition may vary. For example, an optimal treatment may require multiple small injections within a damaged/defective region where no two injections penetrate the same site. Alternatively, the target location may remain the same of successive cell administration procedures.

A suitable catheter system that may be used in the present disclosure is the NOGA™ Navigation Catheter and the MyoStar Injection Catheter. Together, these are part of the NOGA™ Mapping and Injection System (Biosense Webster, Inc.). This catheter is a multi-electrode, percutaneous catheter with a deflectable tip and injection needle designed to inject agents into the myocardium. The tip of the Injection Catheter is equipped with a Biosense location sensor and a retractable, hollow 27-gauge needle for fluid delivery. The injection site is indicated in real-time on the heart map, allowing for precise distribution of the injections. Local electrical signals are obtained to minimize catheter-tip trauma.

### Compositions of the Disclosure

In one example of the present disclosure the mesenchymal lineage precursor or stem cells and/or progeny thereof and/or soluble factor derived therefrom are administered in the form of a composition. In one example, such a composition comprises a pharmaceutically acceptable carrier and/or excipient.

The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. For example, such a media composition does not induce any adverse effects in a subject to whom it is administered.

Exemplary carriers and excipients do not adversely affect the viability of a cell and/or the ability of a cell to reduce, prevent or delay metabolic syndrome and/or obesity.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

The mesenchymal lineage precursor or stem cells and/or progeny thereof and/or soluble factor derived therefrom can also be incorporated or embedded within scaffolds that are recipient-compatible and which degrade into products that are not harmful to the recipient. These scaffolds provide support and protection for cells that are to be transplanted into the recipient subjects. Natural and/or synthetic biodegradable scaffolds are examples of such scaffolds.

A variety of different scaffolds may be used successfully in the practice of the disclosure. Exemplary scaffolds include, but are not limited to biological, degradable scaffolds. Natural biodegradable scaffolds include collagen, fibronectin, and laminin scaffolds. Suitable synthetic material for a cell transplantation scaffold should be able to support extensive cell growth and cell function. Such scaffolds may also be resorbable. Suitable scaffolds include polyglycolic acid scaffolds, (e.g., as described by Vacanti, et al. J. Ped. Surg. 23:3-9 1988; Cima, et al. Biotechnol. Bioeng. 38:145 1991; Vacanti, et al. Plast. Reconstr. Surg. 88:753-9 1991); or synthetic polymers such as polyanhydrides, polyorthoesters, and polylactic acid.

In another example, the mesenchymal lineage precursor or stem cells and/or progeny thereof and/or soluble factor derived therefrom may be administered in a gel scaffold (such as Gelfoam from Upjohn Company).

The compositions described herein may be administered alone or as admixtures with other cells. The cells of different types may be admixed with a composition of the disclosure immediately or shortly prior to administration, or they may be co-cultured together for a period of time prior to administration.

In one example, the composition comprises an effective amount or a therapeutically or prophylactically effective amount of mesenchymal lineage precursor or stem cells and/or progeny thereof and/or soluble factor derived therefrom. For example, the composition comprises about 1×10⁵ stem cells to about 1×10⁹ stem cells or about 1.25×10³ stem cells to about 1.25×10⁷ stem cells/kg (80 kg subject). The exact amount of cells to be administered is dependent upon a variety of factors, including the age, weight, and sex of the subject, and the extent and severity of the disorder being treated.

Exemplary dosages include at least about 1.2×10⁸ to about 8×10¹⁰ cells, such as from about 1.3×10⁸ to about 8×10⁹ cells, for example, from about 1.4×10⁸ to about 8×10⁸ cells, for example, from about 1.5×10⁸ to about 7.2×10⁸ cells, from about 1.6×10⁸ to about 6.4×10⁸ cells, such as from about 1.7×10⁸ to about 5.6×10⁸ cells, for example, from about 1.8×10⁸ to about 4.8×10⁸ cells, for example, from about 1.9×10⁸ to about 4×10⁸ cells, from about 2.0×10⁸ to about 3.2×10⁸ cells, from about 2.1×10⁸ to about 2.4×10⁸ cells. For example, a dose can include at least about 2.0×10⁸ cells. For example, a dose can include at least about 1.5×10⁸ cells.

Expressed another way, exemplary doses include at least about 1.5×10⁶ cells/kg. For example, a dose can comprise from about 1.5×10⁶ to about 1×10⁹ cells/kg, such as from about 1.6×10⁶ to about 1×10⁸ cells/kg, for example, from about 1.8×10⁶ to about 1×10⁷ cells/kg, for example, from about 1.9×10⁶ to about 9×10⁶ cells/kg, from about 2.0×10⁶ to about 8×10⁶ cells/kg, such as from about 2.1×10⁶ to about 7×10⁶ cells/kg, for example, from about 2.3×10⁶ to about 6×10⁶ cells/kg, for example, from about 2.4×10⁶ to about 5×10⁶ cells/kg, for example, from about 2.5×10⁶ to about 4×10⁶ cells/kg, for example, from about 2.6×10⁶ to about 3×10⁶ cells/kg. For example, a dose can include at least about 2.5×10⁶ cells/kg.

In an example, the mesenchymal lineage precursor or stem cells comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

Compositions of the disclosure may be cryopreserved. Cryopreservation of mesenchymal lineage precursor or stem cells can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cryopreservation solution may comprise a sterile, non-pyrogenic isotonic solution such as, for example, PlasmaLyte A™. 100 mL of PlasmaLyte A™ contains 526 mg of sodium chloride, USP (NaCl); 502 mg of sodium gluconate (C₆H₁₁NaO₇); 368 mg of sodium acetate trihydrate, USP (C₂H₃NaO₂.3H₂O); 37 mg of potassium chloride, USP (KCl); and 30 mg of magnesium chloride, USP (MgCl₂.6H₂O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, αMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity. Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises about 5% human serum albumin (HSA) and about 10% DMSO. The cryopreservation solution may further comprise one or more of methycellulose, polyvinyl pyrrolidone (PVP) and trehalose.

In one embodiment, cells are suspended in 42.5% Profreeze™/50% αMEM/7.5% DMSO and cooled in a controlled-rate freezer.

The cryopreserved composition may be thawed and administered directly to the subject or added to another solution, for example, comprising HA. Alternatively, the cryopreserved composition may be thawed and the mesenchymal lineage precursor or stem cells resuspended in an alternate carrier prior to administration.

In an example, the compositions described herein may be administered between about 1 and about 10 days post MI.

In other examples, the compositions described herein may be administered between about 1 and 9 days, between about 1 and 8 days, between about 2 and 7 days, between about 2 and 6 days, between about 3 and 5 days post MI. For example, the compositions described herein may administered about 5 days post-MI.

In an example, the compositions described herein may be administered between about 1 and about 10 days post percutaneous coronary intervention (PCI).

In other examples, the compositions described herein may be administered between about 1 and 9 days, between about 1 and 8 days, between about 2 and 7 days, between about 2 and 6 days, between about 3 and 5 days post PCI. For example, the compositions described herein may administered about 5 days post PCI.

In an example, the compositions described herein may be administered as a single dose.

In some examples, the compositions described herein may be administered over multiple doses. For example, at least 2, at least 3, at least 4, at least 5, at least 6, at least 7, at least 8, at least 9, at least 10 doses.

In one example, the mesenchymal lineage precursor or stem cells can be culture expanded prior to administration. Various methods of cell culture are known in the art.

In an example, mesenchymal lineage precursor or stem cells are culture expanded in a serum free medium prior to administration.

In some examples, the cells are contained within a chamber that does not permit the cells to exit into a subject's circulation but permits factors secreted by the cells to enter the circulation. In this manner soluble factors may be administered to a subject by permitting the cells to secrete the factors into the subject's circulation. Such a chamber may equally be implanted at a site in a subject to increase local levels of the soluble factors, e.g., implanted in or near the heart.

The mesenchymal lineage precursor or stem cells and/or progeny thereof and/or soluble factor derived therefrom cells may be administered systemically, such as, for example, by intravenous, intraarterial, or intraperitoneal administration. The mesenchymal lineage precursor or stem cells and/or progeny thereof and/or soluble factor derived therefrom may also be administered by intramuscular or intracardiac administration.

In an example, the mesenchymal lineage precursor or stem cells and/or progeny thereof and/or soluble factor derived therefrom are administered directly into the myocardium. For example, the mesenchymal lineage precursor or stem cells and/or progeny thereof and/or soluble factor derived therefrom can be administered directly into the myocardium of the left ventricle.

In an example, the mesenchymal lineage precursor or stem cells and/or progeny thereof and/or soluble factor derived therefrom are administered via an endomyocardial catheter such as the J&J Myostar™ injection catheter.

In an example, the mesenchymal lineage precursor or stem cells and/or progeny thereof and/or soluble factor derived therefrom are administered to viable myocardium.

In an example, the mesenchymal lineage precursor or stem cells and/or progeny thereof and/or soluble factor derived therefrom are administered to hibernating myocardium.

One of skill in the art would be able to identify viable and/or hibernating myocardium using methods known in the art. For example, a mapping catheter system such as the NOGASTAR™ Mapping Catheter system can be used to identify viable and/or hibernating myocardium.

In another example, the mesenchymal lineage precursor or stem cells and/or progeny thereof and/or soluble factor derived therefrom are administered via intracoronary infusion. For example, mesenchymal lineage precursor or stem cells and/or progeny thereof and/or soluble factor derived therefrom may be administered into the left anterior descending (LAD) artery.

In an example, mesenchymal lineage precursor or stem cells and/or progeny thereof and/or soluble factor derived therefrom are administered into the LAD artery immediately after LAD revascularisation via PCI.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

The following specific examples are to be construed as merely illustrative, and not limitative of the remainder of the disclosure in any way whatsoever. Without further elaboration, it is believed that one skilled in the art can, based on the description herein, utilize the present invention to its fullest extent.

### EXAMPLES

### Mesenchymal Lineage Precursor or Stem Cells (MLPSCs) Prepared Using Plastic Adherence Techniques

MLPSCs were generated de novo from bone marrow as described in U.S. Pat. No. 5,837,539. Approximately 80-100 ml of bone marrow was aspirated into sterile heparin-containing syringes and taken to the MDACC Cell Therapy Laboratory for MSC generation. The bone marrow mononuclear cells were isolated using ficoll-hypaque and placed into two T175 flasks with 50 ml per flask of MLPSC expansion medium which includes alpha modified MEM (αMEM) containing gentamycin, glutamine (2 mM) and 20% (v/v) fetal bovine serum (FBS) (Hyclone).

The cells were cultured for 2-3 days in 37° C., 5%CO₂ at which time the non-adherent cells were removed; the remaining adherent cells were continually cultured until the cell confluence reached 70% or higher (7-10 days), and then the cells were trypsinized and replaced in six T175 flasks with expansion medium (50 ml of medium per flask).

### Immunoselection of Mesenchymal Lineage Precursor or Stem Cells (MLPSCs)

Bone marrow (BM) was harvested from healthy normal adult volunteers (20-35 years old). Briefly, 40 ml of BM is aspirated from the posterior iliac crest into lithium-heparin anticoagulant-containing tubes.

Bone marrow mononuclear cells (BMMNC) were prepared by density gradient separation using Lymphoprep™ (Nycomed Pharma, Oslo, Norway) as previously described by Zannettino et al., 1998. Following centrifugation at 400×g for 30 minutes at 4° C., the buffy layer is removed with a transfer pipette and washed three times in “HHF”, composed of Hank's balanced salt solution (HBSS; Life Technologies, Gaithersburg, Md), containing 5% fetal calf serum (FCS, CSL Limited, Victoria, Australia).

STRO-3+ (or TNAP+) cells were subsequently isolated by magnetic activated cell sorting as previously described by Gronthos & Simmons, 1995; and Gronthos, 2003. Briefly, approximately 1-3×10⁸ BMMNC are incubated in blocking buffer, consisting of 10% (v/v) normal rabbit serum in HHF for 20 minutes on ice. The cells are incubated with 200 μl of a 10 μg/ml solution of STRO-3 mAb in blocking buffer for 1 hour on ice. The cells are subsequently washed twice in HHF by centrifugation at 400×g. A 1/50 dilution of goat anti-mouse γ-biotin (Southern Biotechnology Associates, Birmingham, UK) in HHF buffer is added and the cells incubated for 1 hour on ice. Cells are washed twice in MACS buffer (Ca²⁺- and Mg²⁺-free PBS supplemented with 1% BSA, 5 mM EDTA and 0.01% sodium azide) as above and resuspended in a final volume of 0.9 ml MACS buffer.

One hundred μl streptavidin microbeads (Miltenyi Biotec; Bergisch Gladbach, Germany) are added to the cell suspension and incubated on ice for 15 min. The cell suspension is washed twice and resuspended in 0.5 ml of MACS buffer and subsequently loaded onto a mini MACS column (MS Columns, Miltenyi Biotec), and washed three times with 0.5 ml MACS buffer to retrieve the cells which did not bind the STRO-3 mAb (deposited on 19 Dec. 2005 with American Type Culture Collection (ATCC) under accession number PTA-7282—see International publication WO 2006/108229, which is incorporated herein by reference in its entirety). After addition of a further 1 ml MACS buffer, the column is removed from the magnet and the TNAP+ cells are isolated by positive pressure. An aliquot of cells from each fraction can be stained with streptavidin-FITC and the purity assessed by flow cytometry.

### Pilot Trial

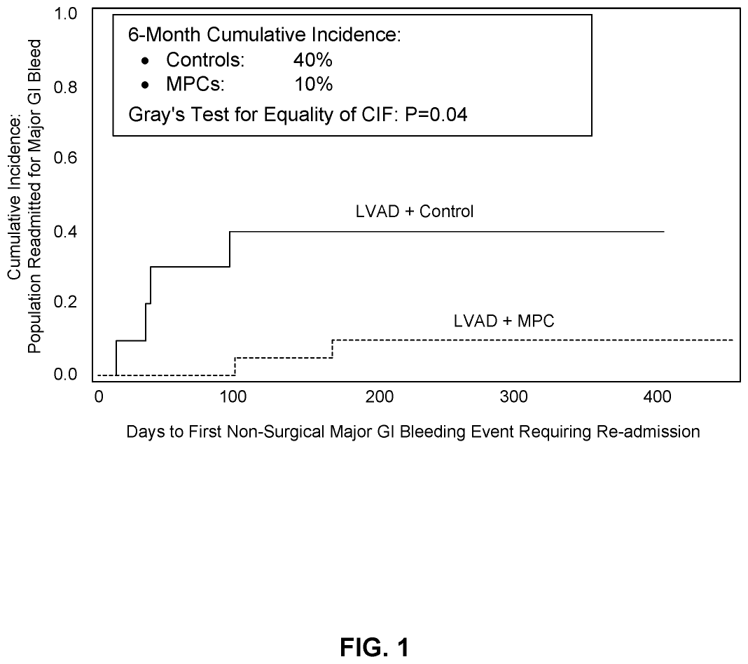

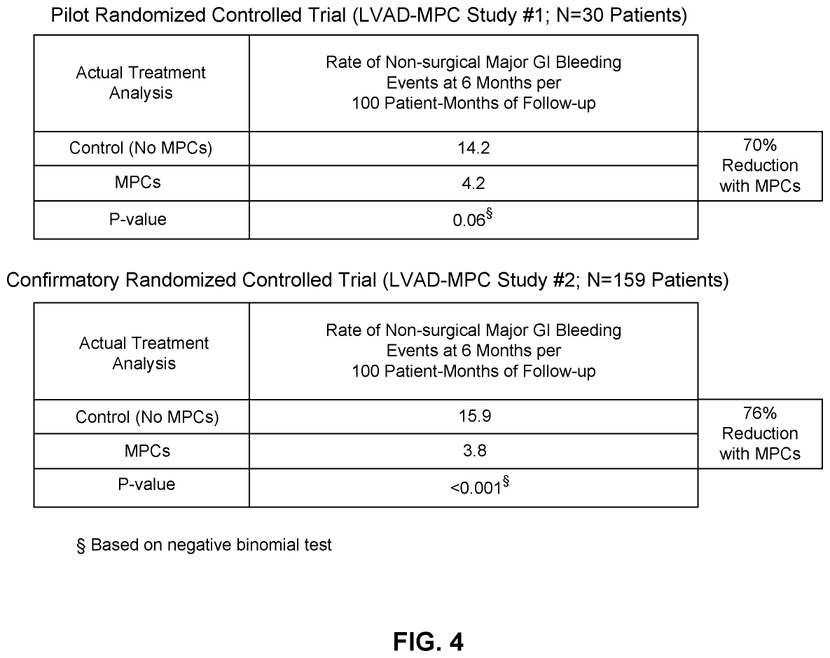

30 patients with LVAD were enrolled in a pilot trial and received MPC or control therapy (LVAD-MPC Study #1). Time to first hospitalisation and overall hospitalisation rates for major GI bleeding were tracked over 12 months. The MPC group had significantly longer time to first hospitalization due to major GI bleeding (p<0.05, Kaplan-Meier statistics; FIG. 1) as well as a 75% reduction in cumulative incidence of at least one hospitalization from GI bleeding observed through 6 months. The percent of patients with at least one hospitalization from GI bleeding observed through 6 months was 16% in MPC group vs 55% in controls, p=0.03 by chi-square test; FIG. 1). A 70% reduction in rate of hospitalizations due to GI bleeding per 100 patient-months of follow-up was also observed in LVAD patients administered MPC's (4.2 in MPC group vs 14.2 in controls, p=0.06 by binomial test; FIG. 4).

### Expanded Confirmatory LVAD-MPC Study #2 Design

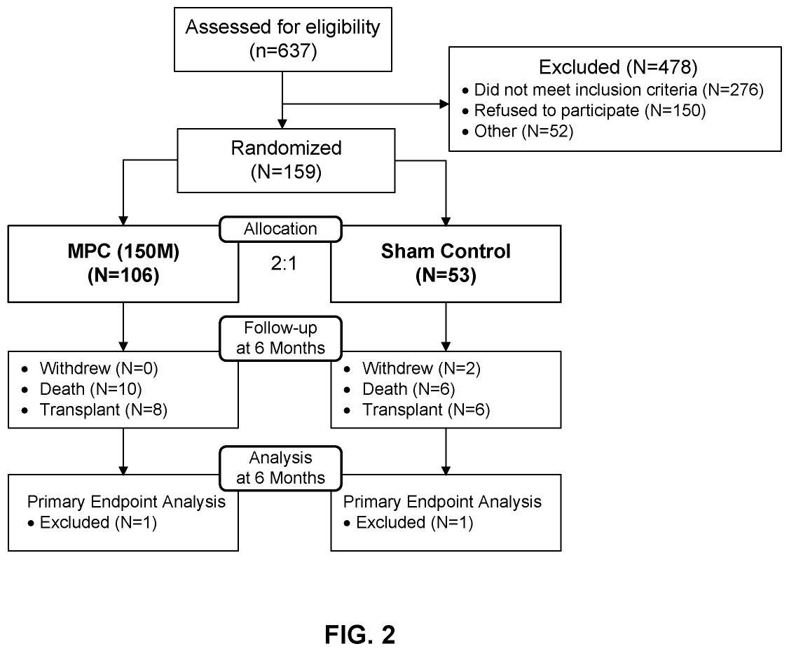

This was a prospective, multi-center, double-blind, randomized (2:1), single dose cohort, sham procedure controlled trial to evaluate the safety and efficacy of injecting a dose of 150 M allogeneic MPCs into the native myocardium of LVAD recipients. The trial design is shown in FIG. 2. Patients with advanced heart failure, implanted with an LVAD as either bridge-to-transplant (BTT) or destination therapy (DT) were eligible to participate in the trial. FDA-approved LVADs were utilized at US sites, and Health Canada-approved LVADs were utilized at Canadian sites. All patients were followed until cardiac transplantation or 24 months post randomization, whichever came first. A total of 159 patients with advanced heart failure who were scheduled to be implanted with an LVAD as a BTT or DT were enrolled in the trial. Assessment of the study outcomes were performed at 60 days, 4 months, 6 months, 9 months, and 12 months post randomization (or until transplant, whichever comes first). Safety was assessed continuously throughout the length of the study.

### Expanded Confirmatory LVAD-MPC Study #2 Results

The results obtained in LVAD-MPC Study #2 showed:

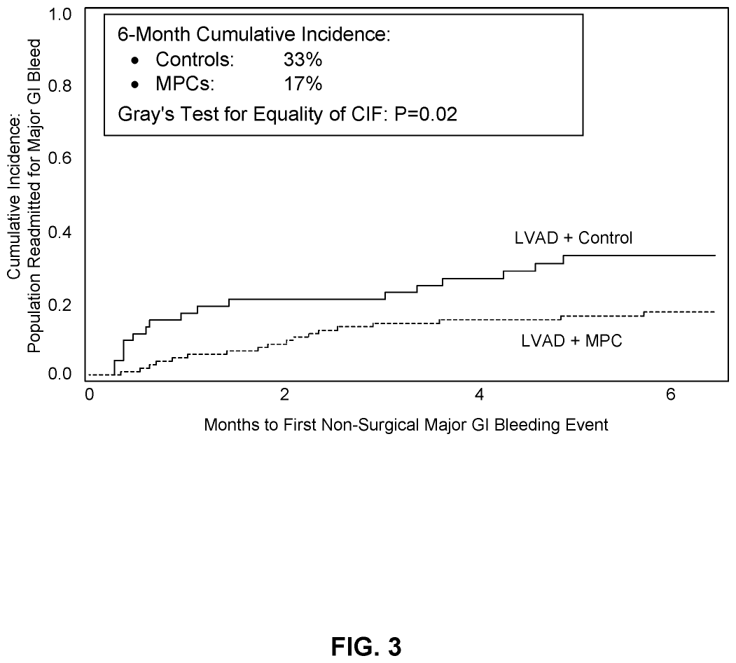

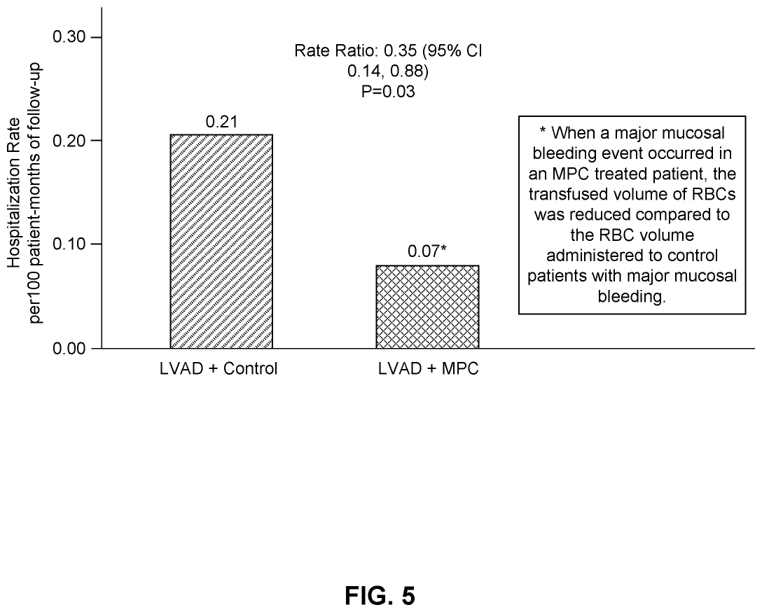

Time to first hospitalisation and overall hospitalisation rates for major GI bleeding were tracked over 6 months. The MPC group had significantly longer time to first hospitalisation due to major GI bleeding (FIG. 3) as well as a 48% reduction in cumulative incidence of at least one hospitalisation from GI bleeding observed through 6 months. A 76% reduction in rate of major GI bleeding events at 6 months per 100 patient-months of follow-up was observed in LVAD patients administered MPC's (3.8 in MPC group vs 15.9 in controls, p, 0.001 by negative binomial test; FIG. 4). In addition, the hospitalization rate for non-surgical major GI bleeding events at 6 months per 100 patient-months of follow-up was reduced by 67% (0.07 in MPC group vs 0.21 in controls [Rate ratio 0.35 (95% CI 0.14, 0.88) p=0.03] (FIG. 5). Of note, when a major GI bleeding event occurred in an MPC treated patient, the transfused volume of red blood cells (RBCs) was reduced compared to the RBC volume administered to control patients with major GI bleeding.

1. A method of treating or preventing gastrointestinal (GI) bleeding in a subject in need thereof, the method comprising administering to the subject a composition comprising mesenchymal lineage precursor or stem cells (MLPSCs).

2. The method of claim 1 wherein the GI bleeding is caused by complications arising from left ventricular assist device (LVAD) implantation in the subject.

3. The method of claim 2 wherein the GI bleeding is associated with inflammation arising from LVAD implantation in the subject.

4. The method of claim 3 wherein the LVAD implantation is a bridge to transplant (BTT) or destination therapy (DT).

5. The method of claim 4 wherein the subject is undergoing treatment or has been treated for heart failure.

6. The method according to claim 6 wherein the heart failure is due to hypertension, cardiomyopathy (ischemic or non-ischemic), myocarditis, obesity, or diabetes.

7. The method of claim 5 wherein the subject has advance ischemic or non-ischemic heart failure.

8. The method according to any preceding claim, wherein the subject has an inflamed myocardium.

9. The method according to any preceding claim, wherein the GI bleeding is associated with aortic stenosis.

10. The method according to any preceding claim, wherein the GI bleeding is associated with von Willebrand's disease.

11. The method according to any preceding claim, wherein the GI bleeding is associated with epistaxis.

12. The method according to any preceding claim, wherein the GI bleeding is associated with a disorder selected from hemorrhoids, peptic ulcers, tears or inflammation in the esophagus, diverticulosis, diverticulitis, ulcerative colitis, Crohn's disease, colonic polyps, and cancer in the colon, stomach or esophagus.

13. The method according to any preceding claim, wherein the cells and/or progeny thereof and/or soluble factors are administered to the subject by a catheter-based system.

14. The method according to claim 9 wherein the cells, and/or progeny thereof and/or soluble factors are administered to the subject by transendocardial injection, intracoronary infusion, intravenous infusion, transepicardial injection. trans-atrial septal or coronary sinus approach.

15. The method according to any preceding claim, wherein the mesenchymal lineage precursor or stem cells and/or progeny thereof and/or soluble factors derived therefrom are administered to the subject between about 1 and 7 days following diagnosis of heart failure.

16. The method according to any preceding claim comprising administering a population of mesenchymal lineage precursor or stem cells enriched for STRO-1+ cells and/or progeny thereof and/or soluble factors derived therefrom.

17. The method according to any preceding claim comprising administering a population of mesenchymal lineage precursor or stem cells enriched for STRO-1^{bright} cells and/or progeny thereof and/or soluble factors derived therefrom.

18. The method according to any preceding claim, wherein the population of mesenchymal lineage precursor or stem cells and/or progeny thereof are administered to the subject or the heart over multiple doses.

19. The method according to any preceding claim comprising administering about 1.5×10⁸ cells and/or progeny thereof.

20. The method according to any preceding claim, wherein the population of mesenchymal lineage precursor or stem cells and/or progeny thereof are administered in the form of a composition together with a pharmaceutically acceptable carrier and/or excipient.
